(19) United States
(12) Patent Application Publication
(10) Pub. No.: US 2010/0249932 A1
(43) Pub. Date: Sep. 30, 2010

(54) Materials, Devices and Methods for Intervertebral Stabilization Via Use of In Situ Shape Recovery

(75) Inventors: Hai H. Trieu, Cordova, TN (US); Jeff R. Justis, Germantown, TN (US); Jeffrey H. Nycz, Warsaw, IN (US)

Correspondence Address: MEDTRONIC;Attn: Noreen Johnson - IP Legal Department, 2600 Sofamor Danek Drive, MEMPHIS, TN, 38132, US

(73) Assignee: WARSAW ORTHOPEDIC, INC., Warsaw, IN (US)

(21) Appl. No.: 12/412,579

(22) Filed: Mar. 27, 2009

(51) Int. Cl. A61F 2/44

(52) U.S. Cl. 623/17.11

(57) ABSTRACT

A deformable implant and a method for expanding an intervertebral disc space are provided. The method may include selecting a deformable implant with a predetermined physical configuration and deforming aspects, including temperature. The deformable implant of some embodiments is heated above the transition temperature or deforming temperature, collapsed to a collapsed configuration and cooled below the transition temperature. The collapsed implant may be inserted into the disc space without distracting adjacent vertebrae. The inserted implant absorbs ambient body heat and expands in place to a final shape thereby distracting adjacent vertebrae to expand the disc space.

### FIELD OF THE INVENTION

The present invention relates to medical devices such as spinal intervertebral implants and methods of use, and more particularly to deformable implants or devices comprised of shape memory material for intervertebral stabilization via in situ expansion of the implant between adjacent vertebral bodies of a spinal column section.

### BACKGROUND

The spine is divided into four regions comprising the cervical, thoracic, lumbar, and sacrococcygeal regions. The cervical region includes the top seven vertebral bodies or members identified as C1-C7. The thoracic region includes the next twelve vertebral members identified as T1-T12. The lumbar region includes five vertebral members L1-L5. The sacrococcygeal region includes nine fused vertebral members that form the sacrum and the coccyx. The vertebral members of the spine are aligned in a curved configuration that includes a cervical curve, thoracic curve, and lumbosacral curve.

Within the spine, intervertebral discs are positioned between the vertebral members and permit flexion, extension, lateral bending, and rotation. An intervertebral disc functions to stabilize and distribute forces between vertebral bodies. The intervertebral disc is comprised of the nucleus pulposus surrounded and confined by the annulus fibrosis. The annulus fibrosus is in turn made up of a series of concentric fiber layers called lamellae.

Intervertebral discs and vertebral members are prone to injury and degeneration. Damage to the intervertebral discs and/or vertebral members can result from various physical or medical conditions or events, including trauma, degenerative conditions or diseases, tumors, infections, disc diseases, disc herniations, scoliosis, other spinal curvature abnormalities or vertebra fractures. In the case of intervertebral discs, damage can also result from normal aging where disc tissue gradually loses its natural moisture and elasticity, causing the disc to shrink or bulge and possibly rupture. In herniated intervertebral discs, damage can occur from normal wear, strain or loading experienced by the disc which causes a disc to tear or rupture.

Damage to intervertebral discs can lead to pain, neurological deficit, and/or loss of motion. Further, damaged intervertebral discs may adversely impact the normal curvature of the spine, and/or lead to improper alignment and positioning of vertebrae which are adjacent to the damaged discs. Additionally, damaged discs may lead to loss of normal or proper vertebral spacing. Various known surgical procedures, treatments and techniques have been developed to address medical problems associated with damaged, abnormal or diseased intervertebral discs.

One common approach to treat a damaged, abnormal or diseased intervertebral disc, is a fusion procedure which removes the damaged disc entirely and fuses the vertebral members which are adjacent to the removed intervertebral disc to prevent relative motion between the adjacent vertebral bodies. The fused vertebral members are typically fused such that there results desired spacing and alignment between the fused vertebral bodies.

A variety of structures can be used to obtain the desired vertebral body spacing and alignment such as spacers, implants or cages. These structures come in a variety of configurations, features, contours, geometries and sizes depending on the specific medical application or use. Further, as is well known to those of skill in the art regarding established surgical procedures, implants can be inserted from a variety of insertion approaches, including anterior, posterior, anterio-lateral, lateral, direct lateral and translateral approaches.

In another approach, a disc augmentation procedure, a section or portion of the damaged intervertebral disc or disc material is removed, and an implant inserted in order to address medical problems associated with damaged diseased intervertebral disc. In this approach some degree of flexibility may remain between the adjacent vertebral bodies.

Surgical implantation and procedures remain difficult and time consuming. A surgeon must always be mindful of the spinal cord and neighboring nervous system. Access to the affected spinal vertebral or disc area may be limited by the person's anatomy. Also, size and configuration of the needed implant or device may present additional obstacles. In some cases, a surgeon may discover that an implanted device has an inappropriate size for a particular application, which requires removal of the implant and insertion of a different size implant. This trial and error approach may increase the opportunity for injury and is time consuming.

One drawback of the techniques discussed above, is the need for distraction of adjacent vertebral bodies to facilitate permit insertion of the implant between the distracted vertebrae. This approach may be problematic where the implant or device has a solid structure and is large which may lead to over distraction of adjacent vertebrae. Over distraction of the vertebral bodies can have a negative impact on the surrounding spinal area and patient anatomy, including nerves, muscles, ligaments, and tissue.

There are known techniques which address over distraction concerns. In one technique, the end plates of adjacent vertebrae are drilled or cut to create an opening or pathway to permit insertion of an implant without distraction. However, this technique can lead to increased possibility of implant expulsion and/or disc annulus damage.

Another known implant delivery technique uses compressive force and moisture content control to facilitate delivery of an implant between adjacent vertebrae, using a polyester type jacket filled with a moisture controlled hydrogel material. In this approach, however, it can be difficult and inconvenient to handle and work with the hydrogel material. There is also the potential of rupture of the implant jacket which can lead to leaked hydrogel in the patient's body. Another implant delivery technique applies mechanical compressive force to physically compress an implant and thereby facilitate delivery of the implant between adjacent vertebrae. However, it can be difficult and potentially unsafe to continuously maintain the required pre-insertion mechanical compressive force.

There is thus a need for improved implants and techniques for expanding the intervertebral disc space while reducing negative consequences of applying distraction forces to adjacent vertebrae. A need exists for an improved intervertebral implant and method for inserting the implant between adjacent vertebral bodies using minimally invasive surgical techniques that overcome drawbacks and difficulties of existing and known implants and insertion techniques.

### SUMMARY

In one aspect of the present invention, there is provided a method for expanding an intervertebral disc space comprising: selecting a deformable implant comprising a predetermined physical configuration and a deforming temperature. Heating the deformable implant to a temperature above the deforming temperature and collapsing the deformable implant to a desired collapsed deformable implant configuration, and cooling the collapsed deformable implant below the deforming temperature to maintain the collapsed deformed implant configuration. Inserting the collapsed deformable implant into the intervertebral disc space. The collapsed deformable implant, absorbing ambient body heat and thereby expanding to a final implant shape within the intervertebral disc space, such that the expandable deformable implant imparts a distraction force on the adjacent vertebrae to obtain an expanded intervertebral disc space.

In another aspect of the present invention, there is provided a method for expanding an intervertebral disc space comprising: selecting a deformable implant comprising a collapsed predetermined physical configuration and a transition temperature. Inserting the collapsed deformable implant into the intervertebral disc space. The collapsed deformable implant, absorbing ambient body heat and thereby expanding substantially to a final implant shape within the intervertebral disc space, such that the expandable deformable implant imparts a distraction force on the adjacent vertebrae to obtain an expanded intervertebral disc space.

Disclosed aspects or embodiments are discussed and depicted in the attached drawings and the description provided below.

### BRIEF DESCRIPTION OF THE DRAWINGS

FIG. 1 illustrates a sagittal plane view of a section of a vertebral column;

FIG. 2 illustrates a view of a deformable implant in a compressed configuration in conjunction with delivery instruments prior to implantation in a damaged vertebral column section according to one embodiment of the present disclosure;

FIG. 3 illustrates a view of the deformable implant post implantation and after initiation of thermal expansion from its collapsed configuration according to one embodiment of the present disclosure; and

FIG. 4 illustrates a view of the deformable implant after expansion and distraction imparted to the adjacent vertebrae according to one embodiment of the present disclosure.

### DETAILED DESCRIPTION

Embodiments of the present invention relate to medical devices such as spinal intervertebral implants and methods of use, and more particularly to deformable implants or devices comprised of shape memory material for intervertebral stabilization via in situ shape recovery or expansion of the implant between adjacent vertebral bodies of a spinal column section. For purposes of promoting an understanding of the principles of the invention, reference will now be made to one or more embodiments, examples, drawing illustrations, and specific language will be used to describe the same. It will nevertheless be understood that the various described embodiments are only exemplary in nature and no limitation of the scope of the invention is thereby intended. Any alterations and further modifications in the described embodiments, and any further applications of the principles of the invention as described herein are contemplated as would normally occur to one skilled in the art to which the invention relates.

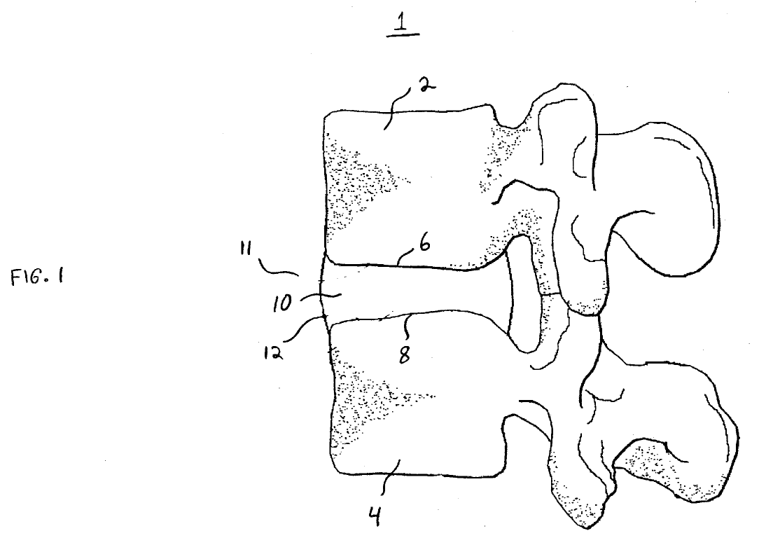

Referring to FIG. 1, there is illustrated a vertebral joint section 1 or motion segment of a spinal vertebral column. The joint section 1 includes adjacent vertebral bodies 2 and 4. The vertebral bodies 2 and 4 include endplates 6 and 8, respectively. An intervertebral disc 10 is located in the intervertebral disc space 11 between the adjacent endplates 6 and 8. The intervertebral disc 10 is comprised of an annulus fibrosus or annulus 12 and a nucleus pulposus in the center of the annulus 12. The annulus 12 extends around a periphery of the intervertebral disc 10. The intervertebral disc 10 substantially occupies the intervertebral disc space 11.

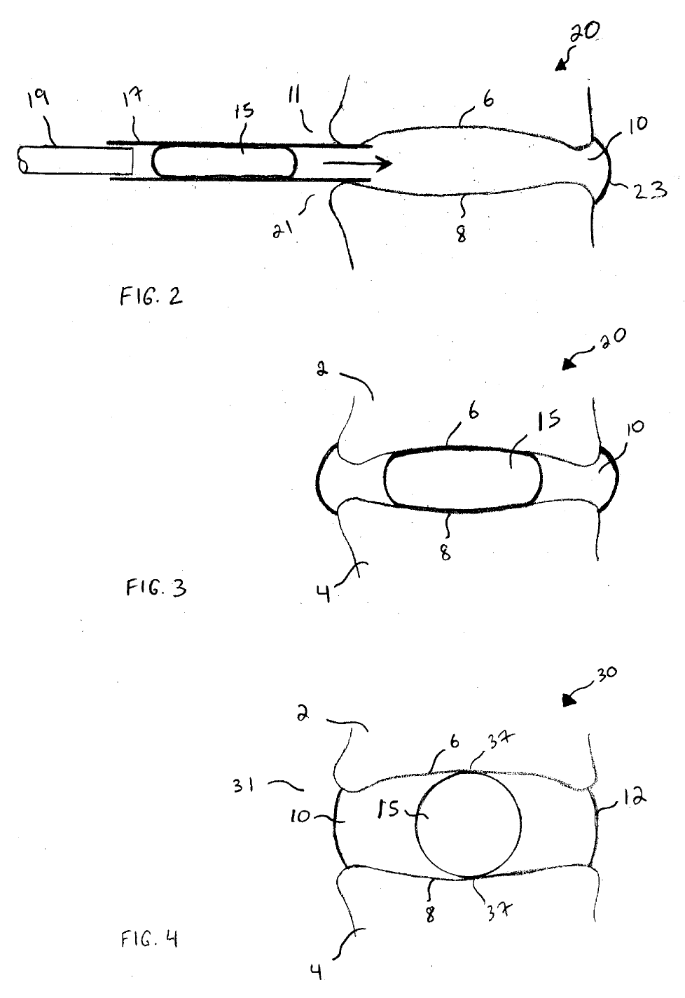

FIGS. 2-4 depict a sequence of views of the implantation and expansion of a deformable intervertebral implant 15 in a vertebral column section according to one preferred embodiment of the present invention. FIG. 2 shows the deformable implant 15 in a compressed configuration in conjunction with a delivery instrument 17 and an insertion instrument 19 prior to implantation or insertion in a damaged vertebral column section 20. FIG. 3 shows the deformable intervertebral implant 15 after implantation or insertion, and after initiation of expansion from its pre-insertion compressed configuration FIG. 4 shows the deformable intervertebral implant 15 after completion of expansion and with distraction imparted to the adjacent vertebrae 2 and 4 via the respective end plates 6 and 8, resulting in an expanded vertebral joint section 30.

Referring to FIGS. 2-4, a deformable intervertebral implant or deformable implant 15 may be used to replace all or a portion of the nucleus pulposus, or to fill all or a portion of the disc space 11. The deformable implant 15 is comprised of a deformable shape memory material which has physical properties or characteristics such that the implant can be deformed or manipulated in response to implant 15 temperature changes. Other aspects also contemplated include a deformable implant 15 comprised of a deformable shape memory material which has physical aspects, properties or characteristics such that the implant can be deformed or manipulated in response to other environmental or ambient factors or changes experience by the deformable implant 15 such as pressure, moisture, vibration, RF energy, light energy, radiation, etc.

In one aspect, a deformable implant 15 is comprised of a shape memory material with a deforming temperature and glass transition temperature (Tg). In one aspect, the deforming or collapsing temperature is greater than the glass transition temperature (Tg). In one aspect the implant can be collapsed, deformed or shaped as desired when the deforming implant temperature is at or above the glass transition temperature (Tg) temperature. And, the collapsed implant will hold its configuration when the implant temperature is below the glass transition temperature (Tg).

The deforming temperature or glass transition temperature (Tg) are aspects of the disclosed deformable implant 15. Depending on the intended medical application, the deformable implant can be manufactured such that the deforming temperature and glass transition temperature (Tg) will have selected values appropriate to the intended medical application use. At the glass transition temperature (Tg), the deformable implant can transition to being compliant or deformable, or less compliant or more rigid. The deforming temperature is not an on/off physical state characteristic, but a temperature starting or inflection transition point for gradual deformable characteristics for the shape deformable implant. It is the temperature at which the implant may be deformed from an original shape to a deformed or collapsed shaped. As the implant temperature increases beyond the glass transition temperature (Tg), the shape memory implant 15 will gradually become more and more compliant and more easily deformable. Conversely, as the implant temperature decreases below the glass transition temperature (Tg), the implant 15 gradually becomes less and less compliant or more rigid as the implant 15 is cooled. The glass transition temperature (Tg) can be viewed as the transition point where the implant is either more compliant and deformable, or less compliant and more rigid depending on whether the implant temperature is changed above or below the glass transition temperature (Tg). Those of skill in the art will recognize that depending on the needed physical implant properties for a specific medical use or application, the glass transition temperature (Tg) can be selectively chosen. The deforming temperature is a temperature at or above the glass transition temperature (Tg) where the shape memory material comprising the implant 15 can be deformed or collapsed transitioned between rigid and compliant states.

At an implant temperature that is below the glass transition temperature (Tg), the memory shape material implant 15 will maintain the shape or configuration it had as it is transitioned below the deforming temperature. As the implant temperature decreases below the glass transition temperature (Tg), the implant 15 gradually becomes less and less compliant or alternatively more and more rigid as the implant 15 is cooled. For example, FIG. 2 illustrates a case where the deformable implant would be at a temperature that is below the glass transition temperature (Tg) and deforming temperature. As such, the implant 15 maintains its collapsed, compressed or deformed shape. In this manner, the deformed implant 15, can be easily and conveniently inserted into a cannula 17 and inserted by an insertion instrument 19 for delivery into the intervertebral disc space 11 without the need to distract the collapsed vertebra 2 and 4.

When the implant temperature is at or above the glass transition temperature (Tg), for example at a deforming temperature that is at or above the glass transition temperature (Tg), the memory shape material implant 15 will have physical characteristics that permit the implant to be manipulated, shaped and compressed such that the implant's original expanded shape (e.g., as shown in FIG. 4) can be deformed, shaped or collapsed so that it can take on a new reduced or collapsed implant profile and configuration (e.g., as shown in FIG. 2). The further away from the glass transition temperature (Tg), the more compliant or deformable the implant 15 will be. In order for the deformed implant to hold its new reduced or collapsed configuration, the temperature of the now deformed implant 15 must be brought down to a point below the deforming temperature or glass transition temperature (Tg). In this manner, the collapsed deformed implant 15 can be conveniently inserted into the cannula 17 or otherwise attached to a delivery instrument for insertion and/or delivery into the intervertebral disc space 11 without the need to distract the collapsed vertebra 2 and 4. The inserted collapsed implant 15 between the vertebral body end plates (shown in FIG. 3) will now gradually absorb heat from its anatomical surroundings. As the collapsed implant 15 absorbs body heat, the implant's 15 temperature will gradually expand to a point near or at the glass transition temperature (Tg) or deforming temperature, which enables the shape memory material implant 15 to transition and expand back to a final shape or substantially back to its original expanded shape (as shown in FIG. 4) between the vertebrae thereby distracting the adjacent vertebrae 2 and 4 and providing a desired or distracted disc space 11. When the implant is in equilibrium with ambient body temperature, it will have a final expanded shape that is either rigid or resilient depending on the desired medical use and application.

The shape memory material implant 15 can be manufactured to have a desired or selected deforming temperature and/or glass transition temperature (Tg). The deforming temperature and glass transition temperature (Tg) can be a specific temperature or range of temperatures which will correspond to a medical application where the implant is to be used. Some spine areas and medical applications contemplated are: intervertebral disc spacers, nucleus replacement implant, disc prosthesis, artificial disc, expandable cage in a variety of shapes, etc. In the case where the shape memory material deforming implant is a fusion device, implant or cage, the deforming temperature and glass transition temperature (Tg) of the implant is selected so that, once inserted in place, the expanded implant will have physical characteristics that result in the implant being or substantially being rigid or hard at body temperature. In this case, the deformable implant must be able to recover its original expanded shape or configuration, and be able to stay rigid at the patient's body temperate so that it wont' collapse when loading is applied to it. In this case, the deforming implant will be manufactured such that it will possess a deforming temperature or glass transition temperature (Tg) is above patient body temperature.

In other medical applications, the shape memory material deforming implant is used for a disc prosthesis, repair or disc augmentation. The deforming temperature and glass transition temperature (Tg) of the implant is selected so that, once inserted in place, the expanded implant final shape, at ambient body temperature, will have physical characteristics that result in the implant being resilient, compliant, and has similar properties and characteristics as those of an intervertebral disc 10. The deformable implant must be able to return to a final shape or substantially back to its original expanded shape or configuration, and be able to maintain resiliency, compliance and motion at patient body temperature similar to an intervertebral disc 10 when loading is applied to it. In this case, the deforming implant may be manufactured such that it will possess a glass transition temperature (Tg) that is near patient body temperature. With this deforming temperature and glass transition temperature (Tg) characteristic, the deformable implant will have the required soft, resilient and compliant properties similar to a normal intervertebral disc at patient body temperature.

In one aspect or embodiment, the glass transition temperature (Tg) is about or near patient body temperature. In a preferred range, glass transition temperature (Tg) can be body temperature ±10° C., where typical body temperature can be 37.0° C.±0.7° C. In another range, glass transition temperature (Tg) can be body temperature ±20° C., 37.0° C.±0.7° C. The deforming temperature will correspond or be selected to the medical application where the implant is to be used. In one aspect, the deforming temperature can be in one or more of the following temperature ranges: an overall range 0° C. (Freezing)-100° C.; a first preferred temperature range near room temperature of 20° C.-50° C.; and a second preferred temperature range near room temperature: 30° C.-40° C. In another application, the deforming temperature can be chosen to be at or near normal body temperature, such as 37.0° C.±0.7° C. In other examples, the deforming temperature and glass transition temperature (Tg) can be selected such that at operating room temperature, at ambient body temperature, or below body temperature, the deformable implant is rigid or substantially rigid. In another example, ambient body temperature, the deformable implant is resilient and somewhat deformable (e.g., as in the case where the deformable implant is inserted and intended to have some resiliency and motion similar to an intervertebral disc).

Further, in addition to selecting the deformable temperature and glass transition temperature (Tg) appropriate for the needed application, as discussed above, the deforming temperature and glass transition temperature (Tg) should also be selected to take into account tools and means that will be used to heat the deforming implant such that the deforming temperature can be reached without undue cost, expense and difficulty. The present disclosures envisions or contemplates heat sources that can heat the deformable implant to and above the deforming temperature or glass transition temperature (Tg), including among others: hot liquid, a heating oven, flame heat, wire resistance heating type devices, an infrared or microwave heat source, or other known heat source mechanisms or devices. These heat sources should apply the necessary heat to the deforming implant in order to raise its temperature to and above deforming temperature or glass transition temperature (Tg) to permit deforming and collapsing of the implant 15.

In addition to the glass transition temperature (Tg) and deforming temperature aspect discussed above, the deformable implant 15 will be manufactured with a desired or original shape or configuration which can be used in a particular medical application. The original or predetermined deformable implant shape can be manufactured to any shape, geometry or configuration selected or requested by a surgeon for a particular medical application. A specific deformable implant shape, geometry or configuration used or selected will depend on the performed medical procedure using the implant. For example, a fusion implant or cage procedure, or disc prosthesis, repair or augmentation procedure. Those of skill in the art will readily recognize that the deformable implant may take on any shaped desired or required for a particular medical use or application.

In one embodiment, shown in FIG. 4, the original shape or configuration can be a spherical or substantially spherical shape or configuration. The shape memory deformable implant 15 can have any geometry or configuration including substantially the following shapes or configurations, among others: spherical, cylindrical, capsule shape, kidney shape, croissant shape, pancake, spherical, cube, toroid, pyramid, polyganol, conic, prism, hemisphere, curved bodies, or other three dimensional configurations. The deformable implant shape may be manufactured and selected to address difficulties inserting or to simplify insertion of an implant into the intervertebral disc space 11. Those of skill in the art will recognize that the novel deformable implant disclosed can be used in any known surgical technique approach for intervertebral medical procedures, including: anterior, anterio-lateral, direct lateral, translateral and Posterior, and known surgical techniques, including among others, open, mini-open and MAST or other minimally invasive surgical techniques.

The selected shape or configuration of a particular deformable implant can be manufactured, machined or molded to have a selected original size and geometry configuration. Also, the selected shape or configuration of a deformable implant can be manufactured or machined from a stock piece or cast stock bar of memory shape material with the selected size and geometry configuration. As previously discussed, the shape memory material implant 15 will also have a desired or selected deforming temperature and glass transition temperature (Tg) for its intended medical application.

In an alternative embodiment, the deformable implant can also be manufactured, machined or cast and then heated to a temperature above its deforming temperature or glass transition temperature (Tg). It is then deformed to a collapsed size and configuration and cooled to hold the collapsed configuration or shape. The collapsed and deformed implant can then be, assuming implant temperature is kept below the deforming temperature or glass transition temperature, shipped in its reduced configuration ready for use in a medical application. This manufacturing approach may be chosen in order to reduce implant preparation time during a surgical procedure and thereby save time and expense.

FIGS. 2-4 show a sequence of views which illustrate an approach that uses the shape memory deformable implant 15 of the present invention to restore appropriate or desired spacing between vertebral bodies 2 and 4 through the implantation and self-expansion of the collapsed deformable intervertebral implant 15 according to a preferred embodiment of the present invention. In one aspect, a shape memory deformable implant 15 with a predetermined physical configuration and a deformable temperature is used. The deformable implant is heated above the glass transition temperature (Tg) to the deforming temperature, then collapsed, deformed or shaped as needed. It is then cooled below the deforming temperature and glass transition temperature (Tg) to maintain the collapsed shape, and inserted in the disc space. The inserted collapsed implant then gradually absorbs ambient body heat to thereby self expand to a final shape or substantially back to its original shape or predetermined physical configuration, thereby imparting a distracting force to the adjacent vertebral bodies to restore desired vertebral body spacing.

In one method of a disclosed embodiment, a deformable implant 15 with a predetermined physical configuration and deforming temperature is selected for use in an intervertebral or other medical procedure. The selected deformable implant 15 can be advantageously used in any intervertebral medical procedure, for example, in one where known polyetheretherketone (PEEK) material implants are currently used. The deformable implant 15 can be used as a fusion implant, spacer or cage, a disc prosthesis, or as a disc repair or augmentation implant. Further, the deformable implant can be manufactured and configured to possess any shaped, geometry or configuration which fits the needs of a particular intervertebral or spinal procedure, use or application.

The deformable implant 15 is first heated so that the implant 15 reaches a temperature above the deforming temperature and/or glass transition temperature (Tg). Any known heat source mechanism can be used to provide the necessary heat, e.g., a hot liquid, a heating oven, heat gun, an electromagnetic wave heat source, or other known heat source mechanisms or devices. The deforming temperature and glass transition temperature (Tg) are predetermined or selected for a specific implant device 15 based on the medical procedure or application where the implant is to be used and the final desired physical structure required. For example, a rigid implant structure in a fusion spacer or cage application, or a flexible resilient implant in a disc prosthesis, repair or augmentation application.

As the shape memory deformable implant temperature is raised to a point at and above the glass transition temperature (Tg) and deforming temperature, the implant's physical body structure gradually begins to exhibit compliant, deformable or flexible physical properties or characteristics.

Having crossed the glass transition temperature (Tg) and deforming temperature threshold, the implant is now amenable to deformation and reshaping. The implant is collapsed, shaped or deformed as needed for the particular implant application and to cooperate with the insertion instruments used in a particular surgical procedure. A shaping or deforming force is applied to deform and shape the heated deformable implant into a desired or needed collapsed insertion physical shape, configuration or geometry. FIG. 2 shows the deformable implant 15 in a collapsed configuration in cooperation with delivery and insertion instruments 17 and 19 prior to insertion into the intervertebral disc space 11 of a damaged vertebral column section 20.

In one aspect, a mold or collapsing instrument with a cylindrical cavity or deforming chamber or means could be used to form the now deformable shape memory material implant 15 into a collapsed configuration in the form of a cylinder or capsule (e.g., as shown in FIG. 2). In the case of FIG. 2, the collapsed cylindrical or capsule implant 15 would be sized during compression to fit inside a cannula 17. The collapsed implant 15 will have a shape and configuration that permits interbody or disc space insertion of the collapsed implant via the cannula 17 and inserter instrument 19 adapted to travel inside the cannula 17. The cannula 17 will be selected to fit between the peripheries 21 of the collapsed vertebral bodies without the need to distract the adjacent vertebrae prior to implant insertion. This aspect eliminates the risk of over distraction of the adjacent vertebral bodies 2 and 4.

Those of skill in the art will recognize that the shaping force can be provided by any device or mechanism that enables the deformable implant to take on a desired collapsed insertion configuration. For example, the implant can be shaped, among others, like a cylinder, capsule, sphere or kidney. Further, the collapsed implant configuration 15 could instead be an implant that is attached to the distal end of a single delivery instrument (e.g., via a known threaded, friction or clamp attachment mechanism) which can insert the collapsed implant directly into the collapsed intervertebral disc space 11.

Once the collapsed deformable implant configuration or shape is obtained, the collapsed implant's temperature is lowered or cooled below the deformable temperature and glass transition temperature (Tg). Below the glass transition temperature (Tg) and deformable temperature, the collapsed deformable implant will maintain or hold the now collapsed shape or configuration, due to its shape memory material properties, without the need for a hold down compressing force. Any known cooling mechanism or device may be used to provide the necessary cooling to lower the implant's temperature below the deforming temperature, e.g., cold water or liquid, refrigeration device, or other known cooling mechanisms or devices.

Alternatively, the selected deformable implant, may be manufactured, heated, collapsed, cooled and then shipped to a surgeon for use in its collapsed form in a medical procedure. In this aspect, the implant is manufactured with a desired physical configuration and deforming temperature and glass transition temperature (Tg). The implant is then heated above its glass transition temperature (Tg) to the deforming temperature, and set to a collapsed configuration or state. The collapsed implant is then cooled so as to maintain its collapsed shape and shipped in its collapsed or deformed state. The shipping package or container must be able to maintain the collapsed implant at a temperature below the deforming temperature and glass transition temperature (Tg) so that the implant can maintain its collapsed configuration. In such an alternative, the surgeon can thereby save time by using a pre-collapsed deformable implant in an intervertebral implant procedure, instead of having to take these steps in the operating room during a surgical procedure.

As a result of the collapsed configuration, the collapsed implant or spacer can be readily and conveniently delivered and inserted into the collapsed intervertebral disc space 11 between the vertebral bodies 2 and 4. The collapsed implant configuration 15 enables the insertion of the collapsed implant 15 without the need to first distract the vertebral body end plates 6 and 8. This aspect eliminates the potential for over distraction of the end plates or the need to cut or drill out a section of the adjacent end plates 6 and 8 for insertion of the implant.

FIG. 2 shows the deformable implant 15 in its collapsed configuration placed inside a delivery instrument (in this case a cannula 17) in preparation for insertion into the collapsed intervertebral disc space 11. The collapsed disc space 11 is also illustrated through the bulging disc annulus at the disc periphery 23. The collapsed implant 15 in the cannula 17 can now be delivered into the collapsed intervertebral disc space 11 via an insertion instrument 19 which travels inside the cannula 17. A delivery force or actuation is then provide to the insertion instrument 19 to force the collapsed implant 15 to move or travel inside the cannula 17 until the implant 15 is inserted into the collapsed intervertebral disc space 11. In this embodiment, the inserted collapsed implant is preferably positioned in the center of the end plates 6 and 8 of the adjacent vertebral bodies, as show in FIG. 3.

The intervertebral disc space 11 may be accessed through a posterior, lateral, translateral, anterior or other suitable surgical approach know to those of skill in the art. Prior to actual insertion, known medical instruments and tools may be used to prepare the intervertebral disc space 11, including specialized pituitary rongeurs and curettes for reaching the nucleus pulposus or other area in the disc space 11. Ring curettes may be used as necessary to scrape abrasions from the vertebral endplates 6 and 8. Using such instruments, a location which will accept the collapsed implant 15 may be prepared in the disc 10 or disc space 11. Those of skill in the art will recognize that the implant may be positioned at any desired location between the adjacent vertebral bodies 2 and 4 depending on the surgeon's need and the performed surgical procedure or medical application.

The now inserted collapsed deformable implant 15 will now gradually absorb ambient patient body heat from the surrounding patient environment. As the collapsed implant 15 absorbs ambient body heat, its temperature will gradually rise. As the collapsed implant warms up and reaches the patient's body temperature, it will begin to gradually thermally expand in place or in situ towards a final shape or substantially back to its original, predetermined implant shape or configuration or to an equilibrium implant configuration. FIG. 3 illustrates the initiated thermal expansion of the collapsed implant 15. At this point, the implant has already absorbed body heat and its temperature is greater than the collapsed implant 15 depicted in FIG. 2. The implant 15 has already begun to gradually expand in place or in situ within the intervertebral disc space 11 in response to the increased implant temperature.

In one aspect of the novel deformable implant, as the collapsed implant gradually warms up and gradually thermally expands, the deformable implant may be manufactured to selectively expand towards a final shape or substantially back to its predetermined implant physical configuration or equilibrium implant configuration in a desired direction or directions within the disc space 31. The implant may be manufactures such that during thermal expansion, the collapsed implant can have a directional expansion aspect or characteristic. The directional expansion within the disc space may be expansion in an axial direction, anterior direction, posterior direction, lateral direction, or a combination of axial and lateral directions, or other desired expansion directions. The implant's directional expansion aspect can be selected or chosen to meets a surgeon's need and/or a specific surgical procedure or medical application.

The collapsed implant 15 will continue to absorb heat and to gradually expand until it is equal to the patient's ambient body temperature. As the device is warming up, it will gradually expand in place between the vertebral body end plates tending to expand towards a final shape or substantially back to its original or predetermined configuration. Alternatively, the expanding implant may take on a final expanded shape at an equilibrium point that is different from the original or predetermined deformable implant shape due to opposing force imparted by the adjacent end plates 6 and 8 against the expanding implant as it expands. Also, if the collapsed implant 15 is expanding in a contoured area between the end plates 6 and 8, then the implant 15 will tend to conform to the shape of the contoured area as it expands. In a preferred aspect, the expanding implant will gradually expand towards a final shape or substantially back to its original shape or predetermined physical configuration, but may also expand such that it compliments the contoured area into which it is expanding. As can be seen in FIGS. 2 and 4, the collapsed implant 15 will expand towards a final shape or substantially back to its predetermined physical configuration such that the height of the expanded implant 15 is greater than the collapsed implant height 15. This results in the expanded or restored intervertebral disc space 31 shown in FIG. 4.

Depending on the medical application for which the implant is used, when the deforming implant reaches body temperature and is fully expanded, the implant will either be rigid or have some resiliency properties depending on whether the medical procedure was for a fusion or disc application. In a fusion application, the expanded implant, now substantially back to its predetermined physical configuration, will be rigid at body temperature and will prevent relative movement between adjacent vertebral bodies. In a fusion application, the glass transition temperature (Tg) can be selected to be close to patient's body temperature, and the deforming temperature can be selected to be above normal patient or human body temperature to thereby permit the deformable implant to maintain a rigid configuration and structure. In a disc application, at patient body temperature, the expanded implant will have flexibility and resiliency to mimic the characteristics or properties of an intervertebral disc. This aspect permits relative motion between the adjacent vertebral bodies. In a disc application, the glass transition temperature (Tg) can be selected to be close to patient's body temperature which permits the deformable implant to retain disc-like flexibility and resiliency in its configuration and structure. In one disc application, the deforming temperature may be near patient or human body temperature. Those of skill in the art will readily recognize that the shape memory implant can be manufactured to have other physical properties, and glass transition temperature (Tg) and deforming temperature which can be selectable to meet a particular surgical need, technique or procedure.

As the collapsed implant 15 continues to absorb ambient heat, it continues to gradually expand in place between the vertebral body end plates 6 and 8 toward a final shape which in some aspects can be substantially back to its original predetermined physical configuration. As implant 15 continues to expand, it thereby imparts a distracting force on the adjacent vertebral end plates 6 and 8. The distracting force pushes the adjacent vertebral bodies 2 and 4 apart or away from each other. FIG. 4 illustrates the deformable implant 15 after completion of its thermal expansion. The thermally expanding implant has imparted a distraction force, via the respective end plates 6 and 8, to the adjacent vertebrae 2 and 4 resulting in an expanded, restored or desired vertebral body spacing 31. The expanded vertebral body spacing 31 results in a disc 10 which no longer has a bulge at its annulus 12 periphery, or has minimal disc bulge. Also in the embodiment shown in FIG. 4, the round or spherical configuration of the deformable implant 15 permits relative spine motion at the interface between the implant and the adjacent vertebral bodies. The expanded or restored disc space 31, and in this case the spherical implant 15, will now alleviate pain or spinal issues that necessitated the intervertebral medical procedure.

The expanded implant 15 will have a final shape. In some aspects, the expanded implant may return substantially back to its original or predetermined implant shape, size and configuration (for example a spherical body as shown in FIG. 4) or it may take on a final shape at an equilibrium point that is different from its original shape or predetermined physical configuration. The inability to fully expand back to its original shape can be due to opposing force imparted by the adjacent end plates 6 and 8 against the expanding implant as it expands between the adjacent vertebra end plates. At an equilibrium point, the adjacent vertebrae counterforce and/or patient anatomy limit how much the collapsed implant 15 can expand in the intervertebral disc space 31, which in turn limits the amount of distraction of the intervertebral disc space 31.

Additionally, the expanded deformable implant 15 may have some subsidence into the adjacent end plates 6 and 8 which have a concave surface configuration. As is well known, the vertebral body end plates 6 and 8 are or can be soft or malleable which may result in end plate subsidence. After implant insertion and completion of the surgical procedure, when a patient gets up and moves around, weight or other contact force is experienced at the interface 37 of the implant's outer surface and the concave end plates 6 and 8. The weight or contact force can result in partial subsidence of the expanded implant body 15 into the concave surfaces of the adjacent end plate 6 and 8 at the contact point or contact area interface 37. Any resulting subsidence further impacts the overall intervertebral disc space 31 distraction that is obtained for a particular expanded implant 15. One positive aspect of the implant's 15 partial subsidence is that the subsidence will act to maintain the expanded deformable implant in place between the vertebral bodies 2 and 4, and prevent it from being ejected out of the vertebral interbody disc space 31 when the patient is active and moves about. Those of skill in the art will recognize that a deformable implant with a predetermined physical configuration must be appropriately selected, in terms of size, configuration and deforming temperature, to account for less than full expansion, end plate subsidence and/or the concave nature of the end plates in order to obtain the desired amount of distraction of the intervertebral disc space 31 or adjacent vertebrae 2 and 4.

While embodiments of the invention have been illustrated and described in detail in the present disclosure, the disclosure is to be considered as illustrative and not restrictive in character. All changes and modifications that come within the spirit of the invention are desired to be protected and are to be considered within the scope of the disclosure.

What is claimed is:

1. A method for expanding an intervertebral disc space comprising:
selecting a deformable implant comprising a predetermined physical configuration and a deforming temperature;
heating the deformable implant to a temperature above the deforming temperature;
collapsing the deformable implant to a desired collapsed deformable implant configuration;
cooling the collapsed deformable implant below the deforming temperature to maintain the collapsed deformed implant configuration;
inserting the collapsed deformable implant into the intervertebral disc space; and
the collapsed deformable implant, absorbing ambient body heat and thereby expanding to a final implant shape within the intervertebral disc space;
wherein the expandable deformable implant imparts a distraction force on the adjacent vertebrae to obtain an expanded intervertebral disc space.

2. The method of claim 1, further comprising cooperatively coupling the collapsed deformed implant configuration to at least one delivery instrument prior to the insertion inserting step.

3. The method of claim 1, wherein the act of inserting is completed without distracting adjacent vertebral bodies.

4. The method of claim 2, wherein the act of coupling comprises inserting the collapsed deformed implant configuration into a delivery instrument.

5. The method of claim 2, wherein the act of collapsing comprises deforming the deformable implant via a delivery instrument.

6. The method of claim 1, wherein the predetermined physical configuration is compliant when deformable implant temperature is at or above the deforming temperature.

7. The method of claim 1, wherein the predetermined physical configuration is rigid when the deformable implant temperature is at or below ambient body temperature.

8. The method of claim 1, wherein the predetermined physical configuration is resilient when the deformable implant temperature is at ambient body temperature.

9. The method of claim 1, wherein the final implant shape is substantially the predetermined physical configuration.

10. The method of claim 1, wherein the collapsed deformable implant expands in an axial direction or lateral direction within the intervertebral disc space.

11. The method of claim 1, wherein the collapsed deformable implant expands in an axial direction and lateral direction within the intervertebral disc space.

12. A method for expanding an intervertebral disc space comprising:
selecting a deformable implant comprising a collapsed predetermined physical configuration and a transition temperature;
inserting the collapsed deformable implant into the intervertebral disc space; and
the collapsed deformable implant, absorbing ambient body heat and thereby expanding substantially to a final implant shape within the intervertebral disc space;
wherein the expandable deformable implant imparts a distraction force on the adjacent vertebrae to obtain an expanded intervertebral disc space.

13. The method of claim 12, further comprising cooperatively coupling the collapsed deformed implant configuration to at least one delivery instrument prior to the insertion inserting step.

14. The method of claim 13, further comprising, prior to the act of coupling,
heating the deformable implant to a temperature above the transition temperature;
collapsing the deformable implant to a desired collapsed deformable implant configuration; and
cooling the collapsed deformable implant below the transition temperature to maintain the collapsed deformed implant configuration.

15. The method of claim 13, wherein the coupling step comprises inserting the collapsed deformed implant configuration into a delivery instrument.

16. The method of claim 13, wherein the act of collapsing comprises deforming the deformable implant via a delivery instrument.

17. The method of claim 12, wherein the predetermined physical configuration is compliant when deformable implant temperature is at or above the transition temperature.

18. The method of claim 12, wherein the predetermined physical configuration is rigid when the deformable implant temperature is at or below ambient body temperature.

19. The method of claim 12, wherein the predetermined physical configuration is resilient when the deformable implant temperature is at ambient body temperature.

20. The method of claim 12, wherein the collapsed deformable implant expands in an axial direction or lateral direction or both axial direction and lateral direction within the intervertebral disc space
